SEQUENCE LISTING (4 sequences)

SEQ ID NO: 1 - protein, 344 aa, Phage P40
MVLVLDISKWQPTVNYSGLKEDVGFVVIRSSNGTQKYDERLEQHAKGLDKVGMPFGLYHYALFEGGQDTINEANMLVSAYKKCRQLGAEPTFLFLDYEEVKLKSGNVVNECQRFIDHVKGQTGVKVGLYAGDSFWKTHDLDKVKHDLRWVARYGVDNGKPSTKPSIPYDLWQYTSKGRIKAIASPVDMNTCSSDILNKLKGSKAPVKPAPKPTPSKPAPAKPAPKTTTKYVNTAHLNIREKASADSKVLGVLDLNDSVQVISESGGWSKLKSGNKQVYVSSKYLSKSKTTPKAKPSSKQYYTIKSGDNLSYIAKKYKTTVKQIQNWNGIKDANKIYAGQKIRVK

SEQ ID NO: 2 - DNA, 1035 bp, Phage P40
atggtattagttttagacatttcaaaatggcaaccgacagtgaattattcaggactaaaagaagatgtaggattcgttgtcattcgttctagcaacggaacacagaagtatgatgagagattagagcaacacgcaaaaggcttagataaagtgggaatgcctttcggactgtaccactacgctttatttgaaggtggacaagatactatcaatgaagcgaatatgttagttagcgcatataagaaatgtcgtcaattaggcgcagaaccaacattcttgttcttagattatgaagaagtcaagttaaaatctggtaatgtggtaaacgaatgtcagagatttatagaccatgtgaaaggtcaaactggggtcaaagtaggactttatgctggggatagtttttggaagacgcacgatttagataaagtcaagcacgatttaagatgggtagctagatatggggtagataacggtaaaccgtctacaaaaccatctataccttatgatttgtggcagtatacttccaaggggcgaattaaagccattgcttcacctgtagatatgaatacatgttctagcgacatattgaacaaattaaaaggttcaaaagcacctgttaaaccagcaccaaaaccgacacctagtaagccagcaccagcgaaaccagcaccaaaaacgactactaaatatgtcaatacggcacatttaaatattcgtgaaaaggcaagtgctgactcgaaagtattgggagttcttgacctcaacgattccgtacaggtcatttctgaatcaggtggatggtctaagttgaaatctgggaacaagcaagtatatgtttctagcaagtatcttagtaagtcaaaaacgacaccgaaggcgaaaccaagctcgaaacagtattatactattaaaagcggtgataatttaagttacattgctaagaagtataaaactacagtaaaacagattcaaaactggaacggtatcaaggatgctaacaaaatttacgcaggtcaaaaaattagagttaaataa

SEQ ID NO: 3 - protein, 200 aa, Phage P40
MVLVLDISKWQPTVNYSGLKEDVGFVVIRSSNGTQKYDERLEQHAKGLDKVGMPFGLYHYALFEGGQDTINEANMLVSAYKKCRQLGAEPTFLFLDYEEVKLKSGNVVNECQRFIDHVKGQTGVKVGLYAGDSFWKTHDLDKVKHDLRWVARYGVDNGKPSTKPSIPYDLWQYTSKGRIKAIASPVDMNTCSSDILNKLK

SEQ ID NO: 4 - protein, 118 aa, Phage P40
TTKYVNTAHLNIREKASADSKVLGVLDLNDSVQVISESGGWSKLKSGNKQVYVSSKYLSKSKTTPKAKPSSKQYYTIKSGDNLSYIAKKYKTTVKQIQNWNGIKDANKIYAGQKIRVK

(19) United States
(12) Patent Application Publication
(10) Pub. No.: US 2014/0079727 A1
(43) Pub. Date: Mar. 20, 2014

(54) NEW ENDOLYSIN PLYP40

(71) Applicants: Martin Loessner, Ebmatingen (CH); Mathias Schmelcher, Schwabhausen (DE)

(72) Inventors: Martin Loessner, Ebmatingen (CH); Mathias Schmelcher, Schwabhausen (DE)

(73) Assignees: Hyglos Invest GMBH; Biomerieux S.A.

(21) Appl. No.: 13/055,646

(22) PCT Filed: Jul. 24, 2009
(86) PCT No.: PCT/EP2009/059606

§ 371 Date: Jan. 6, 2012

(30) Foreign Application Priority Data
Jul. 25, 2008 (DE) 102008002972.6

(51) Int. Cl. C12N 9/24; A61K 38/47; A61K 45/06; A01N 63/00

(52) U.S. Cl. CPC C12N 9/2402; A01N 63/00; A61K 38/47; A61K 45/06
USPC 424/190.1; 435/200; 536/23.2; 435/320.1; 435/252.3; 435/254.2; 435/254.21; 435/7.32; 424/94.61

(57) ABSTRACT

The present invention relates to a polypeptide with an amino acid sequence according to SEQ ID NO:1. The present invention further relates to the nucleic acid molecules comprising a nucleotide sequence coding for the polypeptide, vectors comprising the nucleic acid molecules, and host cells for the expression of the polypeptides. In addition, the present invention relates to the use of the polypeptide as a human medical, veterinary medical or diagnostic substance, as an antimicrobial substance in food, in cosmetics, as disinfecting agent or in the environmental field.

The present invention relates to a polypeptide with an amino acid sequence according to SEQ ID NO:1. The present invention further relates to the nucleic acid molecules comprising a nucleotide sequence coding for the polypeptide, vectors comprising the nucleic acid molecules, and host cells for the expression of the polypeptide. Moreover, the present invention relates to the use of the polypeptide as a human medical, veterinary medical or diagnostic substance, as an antimicrobial agent in food, in cosmetics, as disinfecting agent or in the environmental field.

Listeria are widespread human and animal pathogenic bacteria in the food sector, which elicit the disease pattern of listeriosis. Food products like fish, meat and dairy products are frequently contaminated with listeria. The genus Listeria comprises 6 distinct species with 16 different serotypes. In detail these are L. monocytogenes having the serotypes 1/2a, 1/2b, 1/2c, 3a, 3b, 3c, 4a, 4ab, 4b, 4c, 4d, 4e, 7; L. innocua having the serotypes 3, 6a, 6b, 4ab, U/S; L. ivanovii having the serotype 5; L. seeligeri having the serotypes 1/2a, 1/2b, 1/2c, 4b, 4c, 4d, 6b; L. welshimeri having the serotypes 1/2a, 4c, 6a, 6b, U/S, and L. grayi having the serotype Grayi. The two species L. monocytogenes and L. ivanovii are considered to be pathogenic. A third species, L. seeligeri, is regarded to be nonpathogenic, however there is one case known, in which L. seeligeri caused meningitis in a human being. The remaining species are considered to be nonpathogenic. About 90% of the listeriosis are attributed to L. monocytogenes serovar 1/2a, 1/2b, and 4b (Wing E J & Gregory S H, 2002, Listeria monocytogenes: Clinical and Experimental Update, J Infect Diseases 185 (Suppl 1): S18-S24).

In fact, listeriosis is a rare disease, but it has to be taken very seriously because of the severity of the disease and the high mortality rate. Although only a minimal percentage of the food related diseases is induced by listeria (about 1% in the USA), almost 30% of the annual illnesses with fatal outcome, which are caused by food pathogens, are assigned to this pathogen. Affected are primarily immunosuppressed persons, e.g. elderly people, diabetics, and persons suffering from cancer and/or AIDS. Pregnant women and the unborn child constitute about 25% of all cases of listeriosis patients. Based on their ability to cross the blood brain barrier or the placental barrier, listeria may cause meningitis, encephalitis, abortions, and stillbirths (Wing E J & Gregory S H, 2002, Listeria monocytogenes: Clinical and Experimental Update, J Infect Diseases 185 (Suppl 1): S18-S24; Doyle M E, 2001, Virulence Characteristics of Listeria monocytogenes, Food Research Institute, October 2001).

Listeria are very well adapted to the survival in the environment during the food production. They are tolerant to weak acids and are capable of reproducing at relatively high salt concentrations and at temperatures from 1° C. to 45° C. The main source of infection are articles of food, in particular those which are not heat-treated prior to consumption, such as many dairy products, smoked fish, salted fish, frozen seafood, meat products, salads, and also at an increasing rate convenience products (“ready-to-eat”-products, especially meat products). The contamination by listeria frequently takes place during the processing of food (removal from the cooking container, slicing, decorating, packaging, etc.). In food products, which are produced with the aid of starter cultures and are not heat-treated (e.g., raw milk cheese, salami), a contamination can also take place through the starter cultures or the raw materials themselves or also during ripening and storage. While there is a zero tolerance for L. monocytogenes in ready-to-eat food in the USA, in many European countries, or Canada as well, a contamination with listeria of up to 100 CFU (colony forming units)/g food is permitted for specific food products. Nevertheless, the food products have to be tested for contamination by listeria in all cases. Many of these food products, e.g. seafood, smoked salmon, dairy products or even prefabricated raw food products, have only a limited shelf life. This frequently results in cost-intensive product recalls, if a listeria contamination and a contamination above the permitted limit, respectively, were detected in these products after delivery.

For this reason there is a great interest in providing methods for the detection as well as for decontamination of listeria. Furthermore, uses of antimicrobial substances are important to prevent the growth of listeria on the one hand and to kill already present listeria on the other hand.

EP0781349 describes inter alia the Listeria phage lysin, Ply511, from the phage A511 that can be used for the aforementioned applications. Based on its broad host range against a multitude of Listeria serovars, Ply511 is very well suited, however, it exhibits a relatively low stability, which is an obstacle especially to its application in food products. Thus, Turner et al. (2007, Syst. And Appl. Microbiol., 30, 58-67) point out proteolysis problems in the expression of Ply511 in lactobacilli for the potential use in food products.

EP 1 531 692 B1 describes the Listeria-phage P100 and the Listeria-phage lysin PlyP100 coded by it.

Both Ply511 and PlyP100 have an optimum activity at a weakly alkaline pH, whereas an optimum activity in the neutral and weakly acidic pH range would be important in many applications.

It is therefore the object of the present invention to provide endolysins against listeria, which have a higher stability as well as a higher activity in the weakly acidic range.

This object is solved by the subject-matter defined in the claims.

The following figures serve to illustrate the invention.

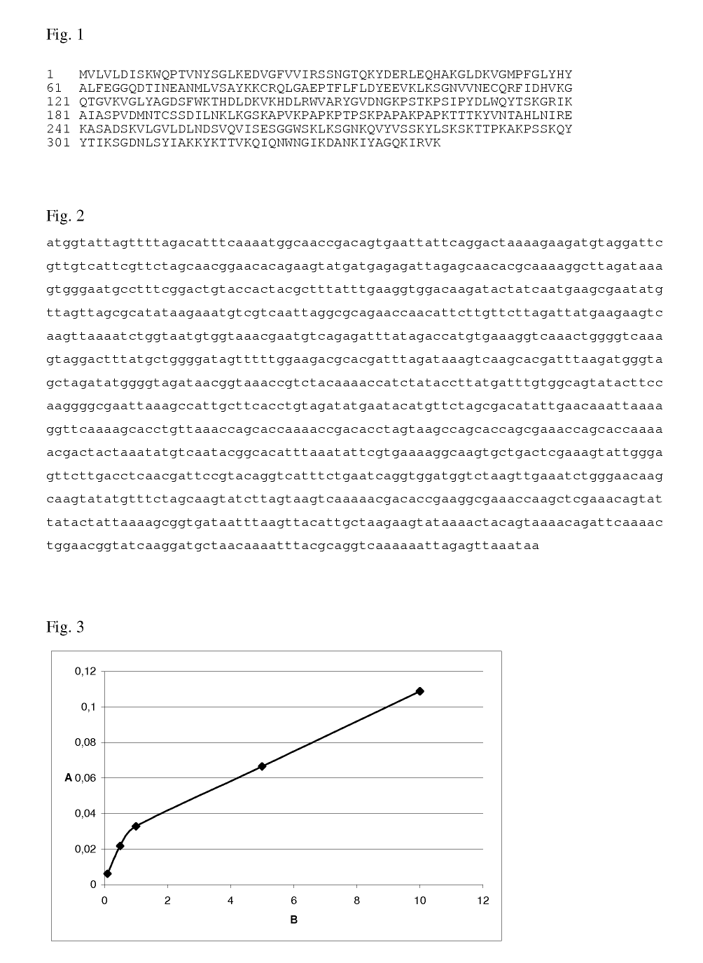

FIG. 1 shows the amino acid sequence of the endolysin PlyP40 (SEQ ID NO: 1) according to the present invention.

FIG. 2 shows the nucleotide sequence (SEQ ID NO:2) coding the endolysin PlyP40 according to the present invention.

FIG. 3 shows in a graph the concentration dependence of the lysis activity of endolysin PlyP40 according to the present invention against L. innocua S1147 (SV 6b). The change in the absorbance per minute (A) was determined as a function of the concentration of PlyP40 (B) in μg/ml.

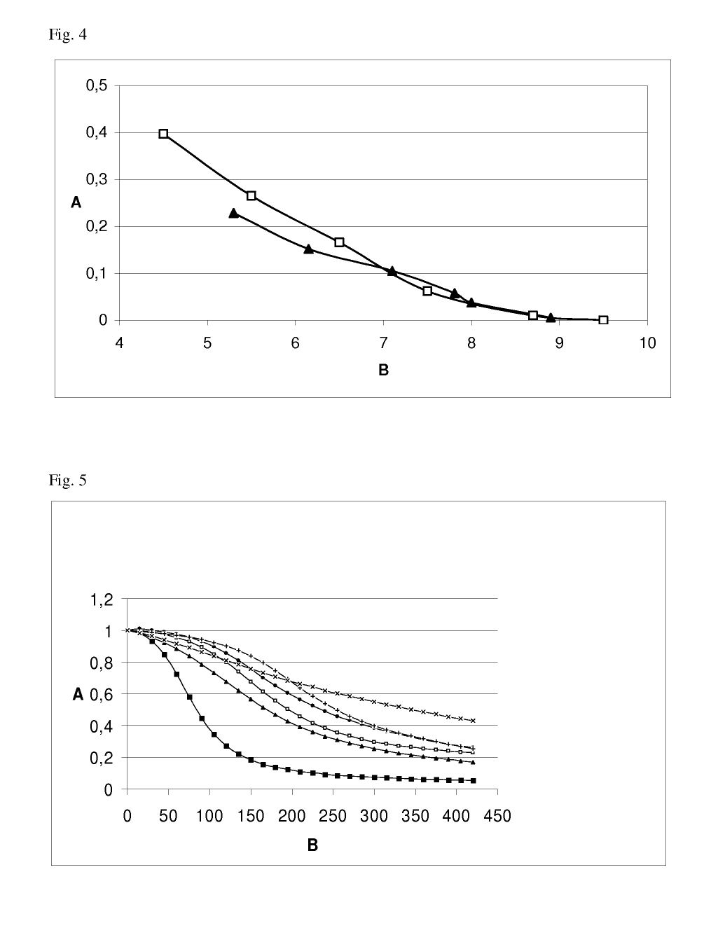

FIG. 4 shows in a graph the pH dependence of the lysis activity of endolysin PlyP40 according to the present invention against L. innocua S1147 (SV 6b). The change in the absorbance per minute (A) was determined as a function of the pH value (B) for 12.8 μg of the endolysin PlyP40 in 1×PBST in the pH range from 5 to 9 (▴) and for 12.8 μg of the endolysin PlyP40 in 50 mM citrate, 50 mM NaH₂PO₄, 50 mM borate in the pH-range from 4.5 to 9.5 (□).

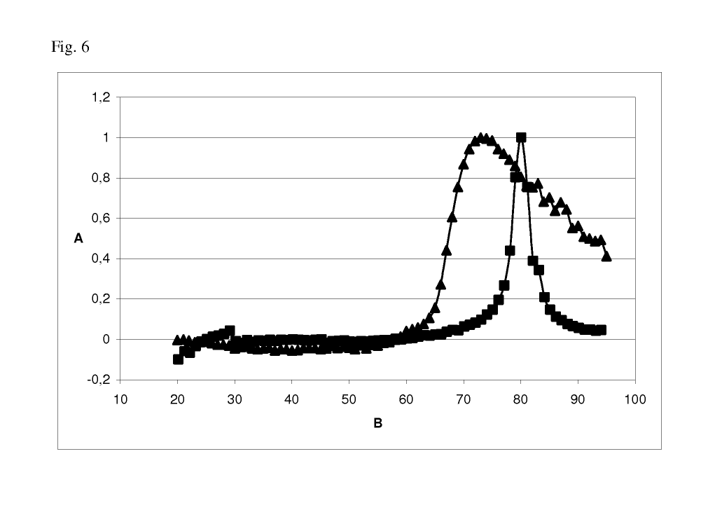

FIG. 5 shows in a graph the activity of the endolysin PlyP40 of the present invention against different Listeria strains. Substrate cells of the Listeria strains L. monocytogenes 1442 SV1/2a (▪), L. monocytogenes 1042 SV 4b (□), L. monocytogenes 1019 SV 4c (), L. monocytogenes 1001 SV 1/2 c (▴), L. innocua 2011 SV 6a (+), and L. welshimeri 50146 SV 6a (x) were normalized to an initial OD₆₀₀ of 1.0. The normalized OD₆₀₀ (A) was traced at a temperature of 30° C. in 1×PBS, pH 8.0, at an initial PlyP40 concentration of 200 pmole/ml as a function of the time (B) in seconds.

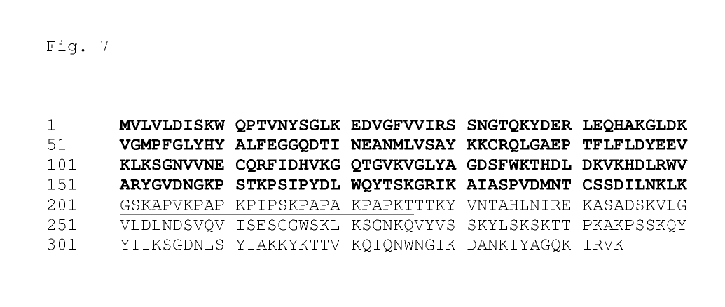

FIG. 6 shows in a graph the analysis of a thermal stability test of the endolysins PlyP40 and Ply511. Endolysin solutions of PlyP40 (▪) and Ply511 (▴) were heated in the photometer. In this process, the increase in the protein aggregation, which corresponds to an increase of the absorbance (A) at a wavelength of 360 nm, is traced as a function of the temperature (B) in degrees Celsius.

FIG. 7 shows the amino acid sequence of the endolysin PlyP40 (SEQ ID NO: 1) according to the present invention. The N-terminal amino acids in bold type at the positions from 1 to 200 represent the enzymatically active domain (EAD). The cell binding domain (CBD) of PlyP40 comprises the C-terminal located amino acids from 227 to 344. The underlined amino acid sequences represent a 26 amino acid linker from position 201 to 226.

The term “listeria” as used herein denotes all bacteria, which are assigned to the genus Listeria. In particular, the term listeria encompasses the species L. monocytogenes having the serotypes 1/2a, 1/2b, 1/2c, 3a, 3b, 3c, 4a, 4ab, 4b, 4c, 4d, 4e, 7; L. innocua having the serotypes 3, 6a, 6b, 4ab, U/S; L. ivanovii having the serotype 5; L. seeligeri having the serotypes 1/2a, 1/2b, 1/2c, 4b, 4c, 4d, 6b; L. welshimeri having the serotypes 1/2a, 4c, 6a, 6b, U/S, and L. grayi having the serotype Grayi.

The term “endolysin”, as used herein, denotes enzymes that are naturally coded by bacteriophages and are produced by them at the end of their host cycle to lyse the host cell and thereby release the offspring phages. Endolysins are comprised of at least one enzymatically active domain (EAD) and a non-enzymatically active cell binding domain (CBD). The EADs can exhibit different enzymatic activities such as, e.g. N-acetyl-muramoyl-L-alanin amidase (amidase, e.g., Ami_{—}2, Ami_{—}5), (endo)-peptidase (e.g., CHAP), transglycosylase, glycosyl hydrolase, (N-acetyl)-muramidase (lysozyme), N-acetyl-glucosaminidase.

The term bacterial “cell wall”, as used herein, denotes all components that form the outer cell enclosure of the bacteria and thus guarantee their integrity. In particular, this refers to the peptidoglycan, the outer membrane of the gram-negative bacteria with the lipopolysaccharide, the bacterial cell membrane, but also to additional layers deposited on the peptidoglycan, like capsules, slimes or outer protein layers.

The term “domain” or “protein domain”, as used herein, denotes a portion of an amino acid sequence that either has a specific functional and/or structural property. On the basis of amino acid sequence homologies, domains can frequently be predicted by employing appropriate computer programs that compare the amino acid sequences in freely available databases with known domains, e.g., Conserved Domain Database (CDD) at the NCBI (Marchler-Bauer et al., 2005, Nucleic Acids Res. 33, D192-6), Pfam (Finn et al., 2006, Nucleic Acids Research 34, D247-D251), or SMART (Schultz et al., 1998, Proc. Natl. Acad. Sci. USA 95, 5857-5864, Letunic et al., 2006, Nucleic Acids Res 34, D257-D260).

The term “domain linker”, as used herein, denotes an amino acid sequence functioning to connect single protein domains with one another. As a rule, domain linkers form no or only few regular secondary structures like α-helices or β-sheets and can occupy different conformations within the respective structural context. Properties of linker sequences as well as methods to detect those are described in the prior art (George & Heringa, 2003, Protein Engineering, 15, 871-879, Bae et al., 2005, Bioinformatics, 21, 2264-2270).

The term “CBD”, as used herein, relates to polypeptide fragments, wherein the respective amino acid sequence corresponds to a portion in endolysins. Said portion is responsible for the binding of the endolysins to the listeria cell wall. Said polypeptide fragments are not enzymatically active. The CBD may also be present as a gene fusion with a spacer molecule (GFP, MBP, biotinylation domains) with and without an affinity tag (His-Tag, Strep-Tag, Avi-Tag, biotinylation domains) or also as a gene fusion only with affinity tag (His-Tag, Strep-Tag, Avi-Tag, biotinylation domains).

The term “EAD” as used herein refers to the enzymatically active domain of a peptidoglycan lysing enzyme which is responsible for hydrolysis of the bacterial peptidoglycan. It contains at least one of the enzymatic activities described for a peptidoglycan lysing enzyme. The term EAD as used herein describes a segment within a polypeptide chain which is derived from a naturally occurring peptidoglycan lysing enzyme.

The term “wild type” or “wt”, as used herein, denotes the amino acid sequence of the endolysin PlyP40 from the phage P40 as specified in SEQ ID NO: 1. The term denotes also the nucleic acid sequence coding the amino acid sequence according to SEQ ID NO: 1. The nucleic acid sequence that codes the endolysin PlyP40 and has been isolated from the phage P40 is specified in SEQ ID NO: 2. The term also encompasses the nucleic acid sequence which contains different codons for single amino acids than those specified in SEQ ID NO: 2, but codes the same amino acid sequence due to the degenerated code.

The term “polypeptide” or “protein”, as used herein, denotes peptides consisting of at least 8 amino acids. The polypeptides can be pharmacologically or immunologically active polypeptides, polypeptides used for diagnostic purposes, or polypeptides used as antimicrobial agent.

The term “protease”, as used herein, denotes an enzyme that is capable of hydrolytically cleave peptide bonds of proteins and/or peptides. The term encompasses proteases, which cleave single amino acids from the amino or the carboxy terminus as well as proteinases which cleave within a protein or polypeptide.

The term “variants”, as used herein, means that a polypeptide has an altered amino acid sequence in comparison to the wild type sequences. The alterations can involve modifications, substitutions, mutations, deletions, additions, and insertions.

The term “mutation”, as used herein, means an alteration of the starting amino acid sequence. Here individual or several immediately consecutive amino acids or amino acids interrupted by non-modified amino acids can be deleted (deletion), added (insertion or addition), or substituted by other amino acids (substitution). The term also encompasses a combination of the individual mentioned alterations. The term encompasses also the N- or C-terminal fusion of a protein or peptide tag.

The term “modification”, as used herein, can be used synonymously with “mutation”. However, the term “modification”, as used herein, also encompasses chemical modifications of the amino acids like e.g., biotinylation, acetylation, chemical modification of the amino-, SH-, or carboxyl groups.

The term “deletion”, as used herein, means the removal of 1, 2 or more amino acids from the respective starting sequence.

The term “insertion” or “addition”, as used herein, means the removal of 1, 2 or more amino acids from the respective starting sequence.

The term “substitution”, as used herein, means the exchange of an amino acid located at a certain position for a different one.

The present invention therefore relates to polypeptides possessing the amino acid sequence according to SEQ ID NO:1.

The endolysin PlyP40 has a length of 344 amino acids in its wild type form. It possesses two functional domains that have only a minimal homology with other known endolysins. The N-terminal amino acids at the positions from 1 to 200 represent the enzymatically active domain (EAD). The cell binding domain (CBD) of PlyP40 comprises the C-terminal located amino acids from 227 to 344. The two domains are connected by a 26 amino acid linker from position 201 to 226.

The present invention further relates to the polypeptides according to the invention comprising modifications. The present invention further relates to the nucleotide sequences coding the polypeptides according to the present invention. The modified polypeptides exhibit the lytic activity of the Wt-PlyP40 endolysin, wherein the activity can be higher, the same or lower, but is not completely lost. The activity is measured with assays known to a person skilled in the art, e.g., the plate lysis assay or the liquid lysis assay.

The modifications can be mutations, in particular deletions, insertions or additions, substitutions or combinations thereof.

Preferably, the deletions introduced into the amino acid sequence according to SEQ ID NO: 1 of the naturally occurring endolysin PlyP40 can shorten the amino acid sequence such that the activity of the protein is not lost. For example, the protease cleavage sites can be removed through the introduced deletions.

The deletions may involve one or several amino acids. When several amino acids are deleted, then the deleted amino acids may be immediately adjacent to each other. Moreover, single deleted amino acids or regions with several deleted amino acids may be separated from each other by one or several non-deleted amino acids. Therefore, one or several deletions may be inserted in the starting sequence of the endolysin PlyP40 according to SEQ ID NO: 1.

Preferably, the substitutions introduced into the amino acid sequence according to SEQ ID NO: 1 of the naturally occurring endolysin PlyP40 can change the amino acid sequence such that the activity of the protein is not lost. For instance, the protease cleavage sites may be altered through the introduced substitutions in such a way that the protease which is specific for the cleavage site does no longer cleave the endolysin.

The substitutions may involve one or several amino acids. When several amino acids are substituted, then the substituted amino acids may be immediately adjacent to each other. Moreover, single substituted amino acids or regions with several substituted amino acids may be separated from each other by one or several non-substituted amino acids. Therefore, one or several substitutions can be inserted in the starting sequence of the endolysin PlyP40 according to SEQ ID NO: 1.

Modifications such as N- or C-terminal tags or chemical modifications of single amino acids may be added to facilitate the production of the proteins (e.g., His-tag or Strep-tag for easier purification), to improve its utilization (e.g., Strep-tag, Avi-tag, JS-tag or chemical biotinylation for the immobilization on surfaces that possess streptavidin or avidin), or enhance solubility or stability (e.g., PEGylation). Furthermore, the modifications can comprise N- or C-terminal HA-tags, Myc-tags or GST-tags. All above mentioned tag-sequences are well known to people skilled in the art. The sequences can be obtained from literature or commercially available vectors.

All of the modified PlyP40 endolysins according to the invention exhibit a lysis activity that is identical or comparable to the naturally occurring PlyP40 endolysin. Furthermore, the above described modifications exhibit positive effects that are beneficial for a commercial application of the endolysins. Such positive effects may involve an enhanced protease stability, thermal stability or stability against chemical denaturing agents. In addition, the stabilization can lead to a higher expression rate, solubility or a longer shelf life. The positive effect may also be expressed by an enhanced activity.

The present invention further relates to polypeptide fragments of the endolysin PlyP40 having the property to bind to the cell wall of listeria, wherein the polypeptide fragments do not exhibit any enzymatically active cell wall hydrolysing regions anymore. Furthermore, the invention relates to the nucleic acid sequences coding for the polypeptide fragments according to the invention. The polypeptide fragments according to the invention are referred to in the following also as “cell wall binding domains” (CBD).

Preferably, the polypeptide fragments according to the invention exhibit an amino acid sequence (referring to the full-length sequence according to SEQ ID NO:1) from about position 227 to 344 as denoted in SEQ ID NO:4. Preferably, the invention relates furthermore to nucleic acid molecules encoding the described preferred polypeptide fragments.

Especially the CBD may be coupled to low molecular substances, e.g., biotin. It may be chemically introduced into the CBD or by fusion of the CBD with a polypeptide, in which biotin is introduced in vivo or in vitro using another protein. Such polypeptides are, e.g., biotinylation domains, i.e., regions in naturally occurring polypeptides, which are biotinylated. Such biotinylation domains are exhibited, e.g., by the oxalacetate decarboxylase of Klebsiella (U.S. Pat. No. 5,252,466 and EP 0511747), the Salmonella typhimurium oxalacetate decarboxylase, the Propionibacterium shermanii transcarboxylase subunit, the biotin carboxyl carrier protein of the Escherichia coli acetyl-CoA carboxylase, the Saccharomyces cerevisiae pyruvate carboxylase or the Saccharomyces cerevisiae acetyl-CoA carboxylase. Such a polypeptide may, however, also be the Avi-Tag (avidity-patents U.S. Pat. No. 5,932,433, U.S. Pat. No. 5,874,239, and U.S. Pat. No. 5,723,584). Furthermore, a biotin may be chemically specifically coupled to a group by fusion with a polypeptide which carries said group, which is not or seldom—but hardly accessible—present in the protein (e.g., cysteine). Furthermore, instead of biotin, the so-called Strep-Tag (Skerra, A. & Schmidt, T. G. M. Biomolecular Engineering 16 (1999), 79-86, U.S. Pat. No. 5,506,121) may be used, which is a short amino acid sequence and binds to streptavidin. Furthermore, the His-Tag may be used. It is also possible to combine different tags and in such a way to use the different binding affinities of the different tags, e.g., Strep-Tag and His-Tag, or biotinylation domain and His-Tag. The biotinylation domains as well as the Avi-Tag, the Strep-Tag as well as the His-Tag are preferably coupled to the CBD using DNA-recombination technology. Preferably, the fusion protein consists of the biotinylation domain of the oxalacetate decarboxylase from Klebsiella or the Avi-Tag, the Strep-Tag or the His-Tag, which are bound to the N-terminal end of the CBD at their C-terminal end. Such a fusion, however, may also be one of the above-mentioned tags, with whose C-terminal end the N-terminus of another protein, which is used as a kind of “spacer molecule”, is coupled, e.g., GFP or maltose binding protein. In this case, the CBD may be coupled via its N-terminal end to the C-terminal end of said other protein.

The CBDs according to the invention may be used for methods for enrichment, removal, and detection of listeria as described in the state of the art.

The present invention further relates to polypeptide fragments of the endolysin PlyP40 having the property to enzymatically hydrolyse the cell wall of Listeria. Furthermore, the invention relates to the nucleic acid sequences coding for the polypeptide fragments according to the invention. The polypeptide fragments according to the invention are referred to in the following also as “enzymatically active domains” (EAD).

Preferably, the polypeptide fragments according to the invention exhibit an amino acid sequence (referring to the full-length sequence according to SEQ ID NO:1) from about position 1 to 200 as denoted in SEQ ID NO:3. Preferably, the invention relates furthermore to nucleic acid molecules encoding the described preferred polypeptide fragments.

Preferably, the invention further relates to nucleic acid molecules comprising nucleotide sequences which code the described modified polypeptides according to the invention. Preferably, a nucleic acid molecule according to the invention comprises a nucleotide sequence according to SEQ ID NO: 2.

The present invention further relates to vectors comprising the nucleic acid molecules according to the invention.

The present invention further relates to appropriate host cells for the expression of the polypeptides according to the invention. Preferably, a suitable host cell for the expression of the polypeptides according to the invention comprises a nucleic acid molecule according to the invention or a vector according to the invention. Preferably, a suitable host cell for the expression of the polypeptide according to the invention is transformed with a nucleic acid molecule according to the invention.

The present invention further relates to the use of the proteins according to the invention as human medical, veterinary medical, or diagnostic substance, as an antimicrobial agent in food or in cosmetics, or as disinfecting agent.

The present invention further relates to a pharmaceutical comprising a polypeptide according to the present invention. The present invention further relates to a pharmaceutical composition comprising the polypeptide according to the present invention. Preferably, a pharmaceutical composition according to the present invention may additionally comprise a pharmaceutically acceptable buffer, a pharmaceutically acceptable diluting agent, or a pharmaceutically acceptable carrier. Moreover, a pharmaceutical composition according to the present invention may contain suitable stabilizing agents, flavors or other suitable reagents.

A further aspect of the present invention relates to the polypeptides according to the invention for the use as human medical, veterinary medical or diagnostic substance for therapy and/or prevention of diseases that are caused by listeria or for the diagnosis of listeria contaminations.

Diseases that are caused by listeria comprise, among others, listeriosis, gastroenteritis, meningitis, encephalitis, sepsis, local wound infections caused by smear infections and inflammations of conjunctiva and cornea.

In a further aspect of the present invention, the polypeptide according to the invention is used in a method for the treatment and/or prophylaxis of infections, in particular of infections that are caused by Listeria. In particular, this Listeria infection can be an infection caused by L. monocytogenes, preferably by L. monocytogenes with the serotypes 1/2a, 1/2b, 1/2c, 3a, 3b, 3c, 4a, 4ab, 4b, 4c, 4d, 4e, 7, especially by L. monocytogenes 1442 SV1/2a, L. monocytogenes 1042 SV 4b, L. monocytogenes 1019 SV 4c and/or L. monocytogenes 1001 SV 1/2 c. In addition, this infection can be a Listeria-infection caused by L. innocua, preferably by L. innocua with the serotypes 3, 6a, 6b, 4ab, U/S, especially by L. innocua 2011 SV 6a. The patient can be a human patient or an animal, in particular animals, which are used in animal husbandry and/or in dairy farming such as ruminants (e.g., cattle, cows, sheep or goats), pigs, horses, poultry, captive wild birds, rabbits, or predators. The method comprises the application of the polypeptides of the present invention in an adequate amount at the site of infection or at a site that is treated prophylactically against an infection.

In a further preferred embodiment, a polypeptide according to the present invention is used in a method for the treatment and/or prophylaxis of gastroenteritis, in particular, gastroenteritis caused by Listeria.

In a further preferred embodiment, a polypeptide according to the present invention is used in a method for the treatment and/or prophylaxis of listeriosis, meningitis, encephalitis, sepsis, as well as local wound infections caused by smear infections and inflammations of conjunctiva and cornea, which are caused especially by Listeria.

In a further preferred embodiment, a polypeptide according to the present invention is used in a method for the treatment and/or the prophylaxis of the above mentioned diseases during prenatal care.

In a particularly preferred embodiment, a polypeptide of the present invention is used for the medical treatment when the infection to be treated or prevented, has been caused by a resistant Listeria strain. Moreover, a polypeptide of the present invention can be used in methods for the treatment of infections through administration in combination with conventional antibacterial active substances, such as antibiotics, other enzymes, e.g., endolysins etc.

The dosage and the type of administration used in a method of treatment and/or prophylaxis of the aforementioned diseases depends on the specific disease and also the site of the infection to be treated. For instance, in particular embodiments of the present invention the type of administration can be an oral, topical, parenteral, intravenous, rectal, or any other type of administration. For the application of a polypeptide of the present invention at the site of infection (or the site at risk of being infected), a polypeptide of the present invention may be formulated in a manner such that the polypeptide is protected from environmental influences like proteases, oxidation, or an immune response etc.

Therefore, a polypeptide of the present invention may be present in a capsule, in a dragee, in a pill, in a suppository, in an injectable solution, or in any other medically suitable galenic formulation. In some embodiments of the present invention, this galenic formulation can contain additionally suitable carriers, stabilizers, flavors, buffers or other suitable reagents.

For instance, for topical applications a polypeptide of the present invention can be administered in the form of a lotion or a plaster.

A suppository formulation may be provided for the treatment of the intestine. Alternatively, an oral administration may be considered. In this case, the polypeptide of the present invention has to be protected from the influences of the gastrointestinal environment until it has reached the site of infection. For example, this can be accomplished through the use of bacteria as carriers, which survive the initial steps of digestion in the stomach and secrete a polypeptide of the present invention later on in the intestinal environment.

All medicinal uses are based on the effect of the polypeptide of the present invention to specifically and immediately lyse Listeria-bacteria when coming into contact with the bacteria. This has an immediate impact on the health status of the treated patient through the reduction of the pathogenic bacteria and bacterial load and the simultaneous support of the immune system. For this purpose, the same galenic formulations can be used such as those that are used in conventional medications for these applications.

In a further aspect, the polypeptides of the present invention are a constituent part of a cosmetic composition. For example, a cosmetic composition according to the invention can be used to inhibit or to prevent irritations caused through an infection of the skin by Listeria bacteria. A cosmetic composition according to the invention preferably contains a sufficient amount of polypeptides according to the invention in order to lyse already existing and/or freshly colonizing Listeria bacteria.

A further aspect of the present invention relates to the use of the polypeptides according to the invention and/or host cells as an antimicrobial substance in food such as dairy products, smoked fish, salted fish, frozen seafood, meat products, salads, and convenience products (“ready-to-eat”-products, especially meat products and ready-made raw food products)

A further aspect of the present invention relates to the use of the polypeptides according to the invention as an antimicrobial substance in food processing devices, in food processing facilities, on surface areas that come into contact with food such as shelves, on containers and in facilities that are used for the storage or the processing of food, and in all other situations, where Listeria bacteria may infest potential food materials. In this context, the polypeptides according to the invention can be used alone or in combination with different antimicrobial substances like disinfecting agents, antibiotics or enzymes such as, for example, different endolysins.

The polypeptides according to the invention can be introduced into or applied to food products and/or at various technical locations within food processing facilities through a multitude of means like for example by admixture of the polypeptides according to the invention to food products, by spraying of the polypeptides according to the invention on facility devices and/or by directly applying the polypeptides according to the invention onto facility devices.

A further aspect of the invention relates to the use of the polypeptides according to the invention in the diagnosis and the detection, respectively, of listeria contaminations in medicine, food industry and food analysis, livestock breeding, and drinking water analysis or environmental analysis.

Listeria contaminations can be detected with the aid of the polypeptides of the present invention in miscellaneous samples like for example in aqueous solutions and mixtures of water and organic solvents, foods, media, blood, blood products, plasma, serum, urine, stool samples, protein solutions, water/ethanol mixtures as well as in solutions in which non-aqueous solid substances to be assayed or isolated, respectively, are dissolved, such as, for example, proteins, DNA, RNA, sugar, salts, food, food-media homogenates, pharmaceuticals, vaccines, organic and inorganic chemicals (e.g., NaCl, MgCl₂, purines, pyrimidines, etc.).

The following examples illustrate the invention and are not to be considered to limit the scope of the invention. Unless stated otherwise, molecular biological standard methods have been used, such as described e.g. by Sambrook et al., 1989, Molecular cloning: A Laboratory Manual, 2nd edition, Cold Spring Harbor Laboratory Press, Cold Spring Harbor, N.Y.

### EXAMPLE 1

### Concentration Dependence of the Lysis Activity of Endolysin PlyP40 Against L. innocua S1147 (SV 6b)

L. innocua S1147 (SV 6b) cells were employed as a substrate in a photometrical lysis assay for the determination of the concentration dependence of the lysis activity of endolysin PlyP40. For this purpose, L. innocua-cells were suspended in PBS buffer with 0.05% Tween 20 at pH 8.0 (1×TBST) and thawed. The change in absorption per minute as an indicator of the lysis activity in dependence of the PlyP40 concentration was determined by adding 1 mM DNase and various amounts of endolysin PlyP40 in a total volume of 1 ml. As a result, a change in absorbance d_{abs}/min per μg of protein in the linear range of 0.0294 was determined, so that a definite activity of the endolysin PlyP40 could be demonstrated.

### EXAMPLE 2

### pH Dependence of the Lysis Activity of Endolysin PlyP40 Against L. innocua S1147 (SV 6b)

To examine the pH dependence of the lysis activity of the endolysin PlyP40, L. innocua S1147 (SV 6b) cells were employed as a substrate in a photometrical lysis assay. For this purpose, frozen L. innocua cells were suspended either in 1×PBS buffers (pH 5-9) or in 50 mM citrate, 50 mM NaH₂PO₄, 50 mM borate buffers (pH 4.5 to 9.5) and thawed. Subsequently, the substrate cell suspensions were mixed with 12.8 μg of endolysin PlyP40 each. The change in the absorption per minute was determined in the photometer as an indicator of the lysis activity. It was found that the maximal lysis activity in both buffer solutions is always found at the lowest pH value examined. A steadily decreasing lysis activity was observed with an increasing pH value, where at a pH greater than 8 only a very low or no lysis activity at all was observed. Hence, the optimum of lysis of endolysin PlyP40 is clearly in the acidic range and thus differs from the optima of lysis of the endolysins Ply511 and PlyP100.

### EXAMPLE 3

### Lysis Activity of Endolysin PlyP40 Against Various Listeria Strains

The lytic activity of endolysin PlyP40 against the Listeria strains L. monocytogenes 1442 SV1/2a, L. monocytogenes 1042 SV 4b, L. monocytogenes 1019 SV 4c, L. monocytogenes 1001 SV 1/2 c, L. innocua 2011 SV 6a, and L. welshimeri 50146 SV 6a was evaluated in a photometric lysis assay. The substrate cells of the individual Listeria strains were added as a starting material at an initial OD600 between 1.0 and 1.5 (normalized to 1.0) in 1×PBS buffer at pH 8 in a total volume of 1 ml. PlyP40 was added at a concentration of 200 pmol/ml at the point in time t=0 and the change in the optical density was traced for several minutes as an indictor of the lysis activity of endolysin PlyP40. The assays were run at 30° C. A high lysis activity of the endolysin PlyP40 was determined for all L. monocytogenes strains from different serovar groups as well as for L. innocua, whereas the lysis activity against L. welshimeri was clearly less pronounced.

### EXAMPLE 4

### Comparison of the Thermal Stability of the Two Endolysins PlyP40 and Ply511

For the test of the thermal stability 100 μg each of endolysin PlyP40 and Ply511 in 25 mM Na-phosphate, 100 mM NaCl, pH 8.0, were placed in a stirrable quartz cuvette (volume 1 ml). The increase in the optical density, which occurs at rising temperatures due to an altered light scattering, caused by the aggregation of the proteins, was traced during heating from 20 to 90° C. A heating rate of 1° C./min was used for heating and the measurement of the optical density was carried out in the photometer at a wavelength of 360 nm. Melting points of 80° C. for endolysin PlyP40 and of 68° C. for endolysin Ply511 were determined by means of the thermal stability assay.

### EXAMPLE 5

### Binding of GFP-Tagged CBDs from Different Listeria Endolysins to the Cell Wall of Different Listeria

Late log phase cells of several Listeria strains were resuspended in PBST (50 mM NaH2PO4, 120 mM NaCl, PH 8.0, 0.01% Tween 20) and incubated with an excess of fusion proteins of green fluorescence protein (HGFP) and CBDs of different listeria binding proteins which are known in the art and described in Korndörfer et al. (2006: The Crystal Structure of the Bacteriophage PSA Endolysin Reveals a Unique Fold Responsible for Specific Recognition of Listeria Cell Walls. J. Mol. Bio. 364: 678-689) and Loessner et al. (2002, Mol Microbiol. 44, 335-349). The respective fusion proteins are incubated for 5 min at room temperature. After washing twice with TBST buffer, the cells were prepared for fluorescence microscopy, using an Axioplan microscope (Carl Zeiss). Pictures of green labeled cells were obtained with a filer set with excitation 450-490 nm, beamsplitter 510 nm, and emission 520 nm. HGFP-CBD-P40 containing amino acid 201 to 344 from SEQ ID NO:1 was C-terminally fused to HGFP as described in Loessner et al., 2002, Mol Microbiol. 44, 335-349. The results of the binding assays are given in table 1 below.

TABLE 1
Binding of GFP-tagged CBDs from different Listeria phage endolysins to the cell
wall of Listeria cells from different species and serovars (++ strong, + weak, (+) very weak, − no binding).

| | WLSC | | | Binding of CBD | | | | | | | |
|---|---|---|---|---|---|---|---|---|---|---|---|
| Species | code | Source | Serovar | 118 | 006 | 500 | PSA | P35 | 511 | P40 | 025 |
| L. monocytogenes | EGDe | J. Kreft | 1/2a | ++ | + | − | − | ++ | ++ | ++ | − |
| L. monocytogenes | 10403S | D. Portnoy | 1/2a | ++ | + | − | − | ++ | + | ++ | − |
| L. monocytogenes | 1442 | Food | 1/2a | ++ | ++ | − | − | − | ++ | ++ | − |
| L. monocytogenes | 1066 | SLCC | 1/2b | ++ | + | − | − | ++ | + | ++ | − |
| | | 8800 | | | | | | | | | |
| L. monocytogenes | 1001 | ATCC | 1/2c | ++ | + | − | − | ++ | + | ++ | − |
| | | 19112 | | | | | | | | | |
| L. seeligeri | 4007 | ATCC | 1/2b | ++ | + | − | − | ++ | + | ++ | − |
| | | 35967 | | | | | | | | | |
| L. welshimeri | 50149 | SLCC | 1/2b | ++ | ++ | − | − | + | ++ | ++ | − |
| | | 5877 | | | | | | | | | |
| L. monocytogenes | 1485 | soft | 3a | + | (+) | − | − | + | + | ++ | − |
| | | cheese | | | | | | | | | |
| L. monocytogenes | 1031 | SLCC1694 | 3b | + | − | − | − | ++ | + | ++ | − |
| L. monocytogenes | 1032 | SLCC | 3c | + | (+) | − | − | ++ | + | ++ | − |
| | | 2479 | | | | | | | | | |
| L. seeligeri | 40127 | SLCC | 3b | + | − | − | − | ++ | ++ | ++ | − |
| | | 8604 | | | | | | | | | |
| L. monocytogenes | 1034 | SLCC | “7” | + | (+) | − | − | − | + | ++ | − |
| | | 2482 | | | | | | | | | |
| L. monocytogenes | 1020 | ATCC | 4a | − | + | ++ | ++ | ++ | ++ | ++ | ++ |
| | | 19114 | | | | | | | | | |
| L. monocytogenes | 1042 | ATCC | 4b | − | − | ++ | ++ | − | + | ++ | − |
| | | 23074 | | | | | | | | | |
| L. monocytogenes | ScottA | J. Jay | 4b | − | − | ++ | ++ | − | + | ++ | − |
| L. monocytogenes | 1019 | ATCC | 4c | − | − | ++ | ++ | ++ | + | + | − |
| | | 19116 | | | | | | | | | |
| L. monocytogenes | 1033 | ATCC | 4d | − | − | ++ | ++ | + | ++ | ++ | − |
| | | 19117 | | | | | | | | | |
| L. monocytogenes | 1018 | ATCC | 4e | − | − | ++ | + | (+) | ++ | ++ | − |
| | | 19118 | | | | | | | | | |
| L. ivanovii | 3009 | SLCC | 5 | − | − | ++ | ++ | ++ | + | ++ | ++ |
| | | 4769 | | | | | | | | | |
| L. ivanovii (ssp. | 3010 | ATCC | 5 | − | − | ++ | ++ | ++ | + | ++ | ++ |
| ivanovii) | | 19119 | | | | | | | | | |
| L. ivanovii (ssp. | 3060 | SLCC | 5 | − | − | ++ | (+) | − | + | ++ | − |
| londoniensis) | | 3765 | | | | | | | | | |
| L. innocua | 2011 | ATCC | 6a | − | − | ++ | (+) | − | + | + | − |
| | | 33090 | | | | | | | | | |
| L. innocua | 2012 | ATCC | 6b | − | − | ++ | ++ | ++ | + | ++ | ++ |
| | | 33091 | | | | | | | | | |
| L. welshimeri | 50146 | SLCC | 6a | − | − | ++ | ++ | + | + | (+) | − |
| | | 7622 | | | | | | | | | |
| L. grayi (ssp. | 6036 | ATCC | − | (+) | − | (+) | − | ++ | (+) | + | − |
| grayi) | | 19120 | | | | | | | | | |
| L. grayi (ssp. | 6037 | ATCC | − | (+) | − | (+) | + | ++ | (+) | + | − |
| murrayi) | | 25401 | | | | | | | | | |

1. A polypeptide comprising an amino acid sequence according to SEQ ID NO:1 or a variant thereof.

2. The polypeptide according to claim 1, wherein the variant has a deletion, addition, insertion and/or substitution in the amino acid sequence according to SEQ ID NO:1.

3. A nucleic acid molecule comprising a nucleotide sequence coding a polypeptide according to claim 1.

4. A vector comprising a nucleic acid molecule according to claim 3.

5. A host cell comprising a nucleic acid molecule according to claim 3.

6. A method for the detection of listeria contamination in a food comprising (a) contacting a polypeptide comprising an amino acid sequence according to SEQ ID NO:1 or a variant thereof with said food; and (b) detecting binding of said polypeptide to listeria in said food.

7. The method according to claim 6, wherein the food comprises a dairy product, a smoked fish, a salted fish, frozen seafood, a meat product, a salad or a convenience product.

8. A method for therapy and/or prevention of disease caused by listeria comprising administering to a subject in need thereof a polypeptide comprising the amino acid sequence according to SEQ ID NO:1 or a variant thereof.

9. The method according to claim 8, wherein the disease caused by listeria comprises listeriosis, gastroenteritis, meningitis, encephalitis, sepsis, local wound infection caused by smear infections or inflammation of conjunctiva or cornea.

10. The method of claim 8, wherein said polypeptide is provided to said subject as prenatal care.

11. A method for the detection of listeria contaminations in a medicine in livestock, in drinking water, in a cosmetic or in an environmental sample comprising (a) contacting a sample with a polypeptide comprising an amino acid sequence according to SEQ ID NO:1 or a variant thereof; and (b) detecting binding of said polypeptide to listeria in a sample or livestock.

12. The method of claim 11, further comprising obtaining a sample from an environment or livestock.

13. A method of disinfecting a food processing device, a food processing facility, a surface area that comes into contact with food, or a facility that is used for storage of food comprising contacting said device, facility or surface with a polypeptide comprising an amino acid sequence according to SEQ ID NO:1 or a variant thereof.

14. The method according to claim 13, wherein the polypeptide is used in combination with another disinfecting agent, antibiotic and/or enzyme.

15. A polypeptide having the sequence according to SEQ ID NO: 3 or 4.

16. A host cell comprising a vector according to claim 4.
